(19) United States
(12) Patent Application Publication
(10) Pub. No.: US 2010/0179653 A1
(43) Pub. Date: Jul. 15, 2010

(54) Intraocular Lenses and Methods of Accounting for Capsule Size Variability and Post-Implant Changes in the Eye

(76) Inventors: Claudio Argento, Los Gatos, CA (US); Terah Whiting Smiley, San Francisco, CA (US); Bryan Patrick Flaherty, Half Moon Bay, CA (US); Barry Cheskin, Los Altos, CA (US)

Correspondence Address: SHAY GLENN LLP, 2755 CAMPUS DRIVE, SUITE 210, SAN MATEO, CA, 94403, US

(21) Appl. No.: 12/685,531

(22) Filed: Jan. 11, 2010

Related U.S. Application Data
(60) Provisional application No. 61/143,559, filed on Jan. 9, 2009.

(51) Int. Cl. A61F 2/16

(52) U.S. Cl. 623/6.13; 623/6.37

(57) ABSTRACT

Accommodating intraocular lenses and methods of use which account for changes to a capsular bag post-implantation as well as a mismatch is size between the accommodating intraocular lens and capsule.

### CROSS-REFERENCE TO RELATED APPLICATIONS

This application claims the benefit of U.S. Provisional Application No. 61/143,559, filed Jan. 9, 2009, which is incorporated herein by reference.

### BACKGROUND OF THE INVENTION

Referring to FIGS. 1 and 2, the structure and operation of a human eye are first described as context for the present invention. Eye 10 includes cornea 11, iris 12, ciliary muscles 13, ligament fibers or zonules 14, capsule 15, lens 16 and retina 17. Natural lens 16 is composed of viscous, gelatinous transparent fibers, arranged in an “onion-like” layered structure, and is disposed in transparent elastic capsule 15. Capsule 15 is joined by zonules 14 around its circumference to ciliary muscles 13, which are in turn attached to the inner surface of eye 10. Vitreous 18 is a highly viscous, transparent fluid that fills the center of eye 10.

Isolated from the eye, the relaxed capsule and lens take on a convex shape. However, when suspended within the eye by zonules 14, capsule 15 moves between a moderately convex shape (when the ciliary muscles are relaxed) and a highly convex shape (when the ciliary muscles are contracted). As depicted in FIG. 2A, when ciliary muscles 13 relax, capsule 15 and lens 16 are pulled about the circumference to a larger diameter, which causes the lens to assume a thinner (as measured along the optical axis) and taller shape. As depicted in FIG. 2B, when ciliary muscles 13 contract, tension in the zonules and capsular bag decreases and the lens assumes a thicker and shorter shape, thus increasing the diopter power of the lens.

The crystalline lens, which is situated posterior of the pupil in a transparent elastic capsule supported by the ciliary muscles, provides about 15 diopters of power, and also performs the critical function of focusing images upon the retina. This focusing ability, referred to as “accommodation,” enables imaging of objects at various distances.

The power of the lens in a youthful eye can be adjusted from 15 diopters to about 29 diopters by adjusting the shape of the lens from a moderately convex shape to a highly convex shape. The mechanism generally accepted to cause this adjustment is that ciliary muscles supporting the capsule (and the lens contained therein), move between a relaxed state (corresponding to the moderately convex shape) to a contracted state (corresponding to the highly convex shape). Because the lens itself is composed of viscous, gelatinous transparent fibers, arranged in an “onion-like” layered structure, forces applied to the capsule by the ciliary muscles cause the lens to change shape.

As a person ages, the lens hardens and becomes less elastic, so that by about age 45-50, accommodation is reduced to about 2 diopters. At a later age the lens may be considered to be non-accommodating, a condition known as “presbyopia.” Because the imaging distance is fixed, presbyopia typically entails the need for bi-focals to facilitate near and far vision.

Cataracts are a major cause of blindness in the world and the most prevalent ocular disease. A cataract is any opacity of a patient's lens, whether it is a localized opacity or a diffuse general loss of transparency. A cataract occurs as a result of aging or secondary to hereditary factors, trauma, inflammation, metabolic or nutritional disorders, or radiation. Age related cataract conditions are the most common. When the disability from cataracts affects or alters an individual's activities of daily living, surgical lens removal with intraocular lens (IOL) implantation is the preferred method of treating the functional limitations.

One method of treating cataracts or a decrease in accommodative ability involves removing the crystalline lens matrix from the lens capsule and replacing it with an intraocular lens (“IOL”). One type of IOL provides a single focal length (i.e., non-accommodating) that allows the patient to have fairly good distance vision. Since the lens can no longer accommodate, however, the patient typically needs glasses for reading.

Apart from age-related loss of accommodation ability, such loss is innate to the placement of IOLs for the treatment of cataracts. After placement of single focal length IOLs, accommodation is no longer possible, although this ability is typically already lost for persons receiving an IOL.

Accommodating IOLs (“AIOL”) function by harnessing the natural force(s) from capsular shape change (in response to zonular tensioning and relaxation) and use it to drive a shape or position change in the AIOL, which in turn adjusts the optical power of the AIOL. The degree of change in optical power of the AIOL depends at least partially on the amount of force imparted to the AIOL from the capsular bag (due to capsular shape change). The degree of accommodation (and/or dis-accommodation) therefore at least partially depends on the degree of engagement between the external surface(s) of an implanted AIOL and the capsular bag. A better “fit” between the AIOL (at least certain portions of the AIOL) and the capsular bag will provide a more efficient transfer of force(s) from the capsular bag to the AIOL.

It is generally desirable to know the size (e.g., diameter, circumference, depth, etc.) of the capsular bag before implanting the AIOL. Additionally, the diameter of a capsule can vary from patient-to-patient or even from eye-to-eye, with the difference in diameter between small diameter capsules and large diameter capsules being roughly about 1.5 mm, or 1500 microns. The fit between the AIOL and the capsule will therefore depend on the patient's measured capsule size. If, for example, the capsular bag is much larger than the AIOL (and therefore does not have a good “fit” with the lens), much of the force(s) a capsular bag is capable of generating can be wasted when the capsular bag changes shape but does not make contact with the AIOL (or does make contact with the IOL but does not apply enough force(s) to the AIOL), which can result in little or no accommodation. Conversely, if the AIOL is larger than the capsular bag and needs to be squeezed into the bag during implantation, the bag will exert a force on the AIOL even in the absence of ciliary muscle contraction. The AIOL may, in some instances, shift to a permanently accommodated configuration, even when the ciliary muscles are relaxed, thereby creating a myopic shift in the patient.

The capsular bag dimensions remain, however, difficult to precisely measure. Current methods of measuring the capsule diameter are only accurate to about +/−300 microns. A risk therefore exists, even after measuring the capsule, that an AIOL will be implanted whose diameter is not desirable based on the actual diameter of the capsule. For example, the implanted AIOL may be too large relative to the actual size of the capsule. This can result in a permanent myopic shift.

Additionally, changes can occur within the eye after lens implantation, or even to the IOL after implantation. For example, it has been noted that there is a healing response (which can vary from patient-to-patient) from the capsule after implantation in which the lens capsule contracts, or shrinks, around the IOL. This is considered to be a fibrotic response from the capsular bag in response to the removal of the native lens from the capsule. The capsular contraction can deform the IOL or portions of the IOL after implantation, which can change the optical power of the IOL. The set-point of the IOL can therefore be affected post-implant by changes that occur in the eye, such as capsular contraction.

One option for accounting for these changes in the eye or to the lens itself after implantation is to make a post-implant adjustment, either to the lens or to a portion of the eye. Some post-implant adjustments require intervention, while some IOLs are configured and arranged to self-adjust, or automatically adjust, post-implant to account for changes that occur within the eye or changes that occur to the lens. Exemplary lenses and post-implant adjustments that can be made to the eye include those described in U.S. application Ser. No. 10/358,038, filed Feb. 2, 2003, U.S. application Ser. No. 10/890,576, filed Oct. 7, 2004, U.S. application Ser. No. 11/507,946, filed Aug. 21, 2006, U.S. application Ser. No. 12/178,304, filed Jul. 23, 2008, U.S. application Ser. No. 10/360,091, filed Feb. 6, 2003, U.S. application Ser. No. 10/639,894, filed Aug. 12, 2003, U.S. application Ser. No. 11/284,068, filed Nov. 21, 2005, U.S. Provisional Application No. 60/402,746, filed Aug. 12, 2002, U.S. Provisional Application No. 60/405,471, filed Aug. 23, 2002, U.S. Provisional Application No. 60/487,541, and U.S. application Ser. No. 10/231,433, filed Aug. 29, 2002, all of which are incorporated by reference herein.

One potential drawback to some post-implant modifications is that they require a second intervention (i.e., an additional step or procedure after the IOL is positioned within the capsular bag). Selecting an IOL for implantation which can automatically account for changes that occur within the eye or to the IOL itself after implantation can potentially avoid requiring a second intervention, which can shorten and/or simply the overall implantation process.

Considering the issues set forth above, a need therefore exists for additional lenses and methods of selecting and implanting an appropriate lens taking into consideration the variability in capsule sizes, the current imperfect techniques for measuring capsule sizes, and/or to account for post-implant changes that can occur within the eye or to the intraocular lens.

### SUMMARY OF THE INVENTION

One aspect is an accommodating intraocular lens (“AIOL”) which includes an optic portion and a peripheral portion, a fluid disposed within at least one of the optic portion and the peripheral portion, wherein the AIOL has a non-linear power response to increasing amounts of capsular force.

In some embodiments a power change of the AIOL during a second portion of the non-linear response is substantially greater than a power change of the AIOL during a first portion of the non-linear response. The power change during the first portion of the non-linear response can be greater than zero.

In some embodiments the power change of the AIOL during a first portion of the non-linear response is substantially zero.

In some embodiments the peripheral portion defines a fluid chamber in fluid communication with a fluid channel within the optic portion, wherein the fluid is disposed within the fluid chamber and the fluid channel. The fluid pressure in the active channel can increase from a first pressure to a second pressure during a first portion of the non-linear response, and the fluid pressure can increase from the second pressure to a third pressure during a second portion of the non-linear response. The fluid pressure in the active channel can also remain substantially the same during the first portion and increase from a first pressure to a second pressure during the second portion

In some embodiments the optic portion comprises an anterior element, a posterior element, and an intermediate element disposed between the anterior element and the posterior element, wherein the intermediate element deflects in response to capsular forces. In some embodiments the intermediate element comprises an actuator which deflects in response to capsular forces on the AIOL. In some embodiments the intermediate element deflects during a first portion of the non-linear response and during a second portion of the non-linear response. The intermediate element may not be in contact with the anterior element at the beginning of the first portion, and is in contact with the anterior element at the beginning of the second portion. In some embodiments a curvature of the anterior element changes more during the second portion than during the first portion. The intermediate element and posterior element can define an active channel in fluid communication with the peripheral portion, wherein the anterior element and the intermediate element define a passive chamber, and the fluid is a first fluid disposed within the active channel and peripheral portion, and wherein the passive chamber contains a second fluid.

In some embodiments the peripheral portion comprises a haptic which deforms in response to capsular bag forces

One aspect is an accommodating intraocular lens (“AIOL”) including an optic portion and a non-optic portion peripheral to the optic portion, wherein the optic portion comprises an actuation element that changes configuration in response to capsular forces on the AIOL, and wherein the AIOL has a non-linear power response in response to capsular forces on the AIOL.

In some embodiments the optic portion comprises an anterior element and a posterior element, and the actuation element is disposed between the anterior element and the posterior element. The actuation element may not be in contact with the anterior element at the beginning of a first portion of the non-linear response, but is in contact with the anterior element at the beginning of a second portion of the non-linear response. The curvature of the anterior element may be adapted to deform in response to capsular forces, and wherein the curvature of the anterior element deforms more during a second portion of the non-linear response than during a first portion of the non-linear response.

In some embodiments the power of the AIOL changes substantially less during a first portion of the non-linear response than during a second portion of the non-linear response.

In some embodiments the power of the AIOL during a first portion of the non-linear response remains substantially constant.

In some embodiments the AIOL further comprises a fluid disposed within at least one of the optic portion and the peripheral portion, wherein the actuation element is adapted to change configuration in response to fluid displacement within the AIOL.

One aspect is a method of accounting for capsular forces on an accommodating intraocular lens. The method includes providing an accommodating intraocular lens (“AIOL”) with an optic portion and a peripheral portion, implanting the AIOL within an eye, and allowing the AIOL to have a non-linear power response to capsular forces on the AIOL while allowing an actuation element within the optic portion to change configurations.

In some embodiments allowing the actuation element within the optic portion to change configurations comprises allowing an actuation element disposed between an anterior element of the optic portion and a posterior element of the optic portion to deflect towards the anterior element or the posterior element. The allowing step can comprise allowing the actuation element to move towards the anterior element or the posterior element without engaging the anterior element or the posterior element during a first portion of the non-linear response. The allowing step can also include allowing the actuation element to engage the anterior element or the posterior element during a second portion of the non-linear response.

In some embodiments providing a plurality of power change phases in response to capsular forces on the AIOL includes providing a first portion of the non-linear response during which the power of the AIOL changes substantially less than during a second portion of the non-linear response. The power of the AIOL can remain substantially the same during the first portion of the non-linear response.

In some embodiments the allowing step comprises allowing a curvature of an anterior element to change more during a second portion of the non-linear response than during a first portion of the non-linear response.

One aspect is a kit of accommodating intraocular lenses. The kit includes a plurality of accommodating intraocular lenses each of which comprises an optic portion and a peripheral portion, wherein each of the plurality of accommodating intraocular lenses has an optic portion element with a different physical parameter. The different physical parameter can be a dimension of the optic portion component. The optic portion component can be an actuator disposed between an anterior surface and a posterior surface of the optic portion.

One aspect is a method of selecting an accommodating intraocular lens for implantation. The method includes measuring a capsular bag characteristic, selecting an accommodating intraocular lens, based at least in part on the measured characteristic, from a plurality of accommodating intraocular lenses, wherein each of the accommodating intraocular lenses has an optical portion element with a different physical parameter, and implanting the accommodating intraocular lens within a patient's eye.

In some embodiments the selecting step comprises selecting an accommodating intraocular lens with the physical parameter which will provide a non-linear power response to capsular forces on the intraocular lens.

One aspect is a method of accommodating an intraocular lens (“AIOL”). The method includes providing an AIOL that changes power during a first portion of a non-linear power response to a first type of ciliary muscle movement, and wherein the AIOL changes power during a second portion of the non-linear response to a second type of ciliary muscle movement, wherein the first and second types of ciliary muscle movements are the same types of movement, and wherein the power change during the first portion of the non-linear power response is different than the power change during the second portion of the non-linear power response. The method also includes implanting the accommodating intraocular lens within a patient's eye to provide an implanted AIOL with a non-linear power response.

In some embodiments the power change during the first portion is substantially less than during the second portion, and wherein the first portion occurs before the second portion. There may be substantially no power change during the first portion.

In some embodiments the AIOL comprises a surface element, wherein the degree of change in the curvature of the surface element during the first portion is different that the degree of change in curvature of the surface element during the second portion.

In some embodiments the first and second types of ciliary muscle movement are ciliary muscle contraction.

One aspect is a method of accommodating an accommodating intraocular lens (“AIOL”). The method includes providing an AIOL that has a non-linear power change response to a singular type of ciliary muscle movement, implanting the AIOL in a patient's eye, and allowing the AIOL to accommodate in response to the singular type of ciliary muscle movement in a non-linear manner.

In some embodiments the singular type of ciliary muscle movement is ciliary muscle contraction.

One aspect is an accommodating intraocular lens including an optic portion and a peripheral portion, wherein the accommodating intraocular lens has a non-linear power change response to a singular type of ciliary muscle movement.

One aspect is an accommodating intraocular lens including an optic portion, a peripheral portion, and a fluid disposed within the optic portion and the peripheral portion, wherein the optic portion and the peripheral portion are in fluid communication, wherein the peripheral portion deforms in response to capsular reshaping due to ciliary muscle movement to displace the fluid between the peripheral portion and the optic portion, and wherein the peripheral portion is configured such that substantially no fluid is displaced between the peripheral portion and the optic portion in response to a non-ciliary muscle movement related capsular reshaping.

In some embodiments the peripheral portion comprises at least one haptic in fluid communication with the optic portion, wherein the haptic is configured to deform in response to non-ciliary muscle movement related capsular reshaping such that substantially no fluid is displaced between the peripheral portion and the optic portion.

In some embodiments a dimension of the at least one haptic is greater than a dimension of a capsule into which the AIOL is implanted.

In some embodiments the peripheral portion includes at least one haptic which has an oval cross-sectional shape.

One aspect is a method of delivering a two part accommodating intraocular lens (“AIOL”). The method includes delivering a frame element within a patient's capsule such that the frame engages and reshapes the capsule, and delivering an AIOL within the capsule to a position to allow the AIOL to accommodate in response to ciliary muscle movement.

In some embodiments delivering the frame comprises allowing the frame to reconfigure from a delivery configuration to an implanted configuration.

In some embodiments reconfiguring the capsule comprises extending the capsule in an axially directed direction. Extending the capsule in an axially directed direction can comprise extending an anterior portion of the capsule in an anterior direction and extending a posterior portion of the capsule in a posterior direction.

In some embodiments delivering the frame element comprises preventing the capsule from applying forces on the AIOL due to non-ciliary muscle movement related capsular forces.

In some embodiments the method does not include securing the frame element to the AIOL.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1-2B illustrate the partial anatomy of an eye.

FIGS. 3-5 illustrate an exemplary accommodating intraocular lens without a dead zone.

FIG. 6 shows a partial cross-section of an exemplary accommodating intraocular lens without a dead zone.

FIG. 7 shows an exemplary accommodating intraocular lens with a dead zone.

FIGS. 8-10 illustrate an exemplary accommodating intraocular lens with a dead zone throughout a range of accommodation.

FIG. 11 shows an exemplary accommodating intraocular lens including a dead zone.

FIGS. 12-14 illustrates an accommodating intraocular lens which can compensate for capsular forces not related to ciliary muscle movement.

FIGS. 15 and 16 illustrate an accommodating intraocular lens which includes haptics with varying stiffness throughout the haptics.

FIGS. 17-19 illustrate accommodating intraocular lenses which are more response to zonular tensioning in zonular contact zones than in non-zonular contact zones.

FIGS. 20-22 illustrates a capsular tensioning frame which can be positioned within a capsule before an accommodating component of the replacement lens.

FIGS. 23-25 illustrate an exemplary lens implanted in a capsule in which a tensioning frame has been positioned.

FIGS. 26A-26C illustrate a cross-section of an exemplary intraocular lens superimposed on cross-sections of three exemplary capsules.

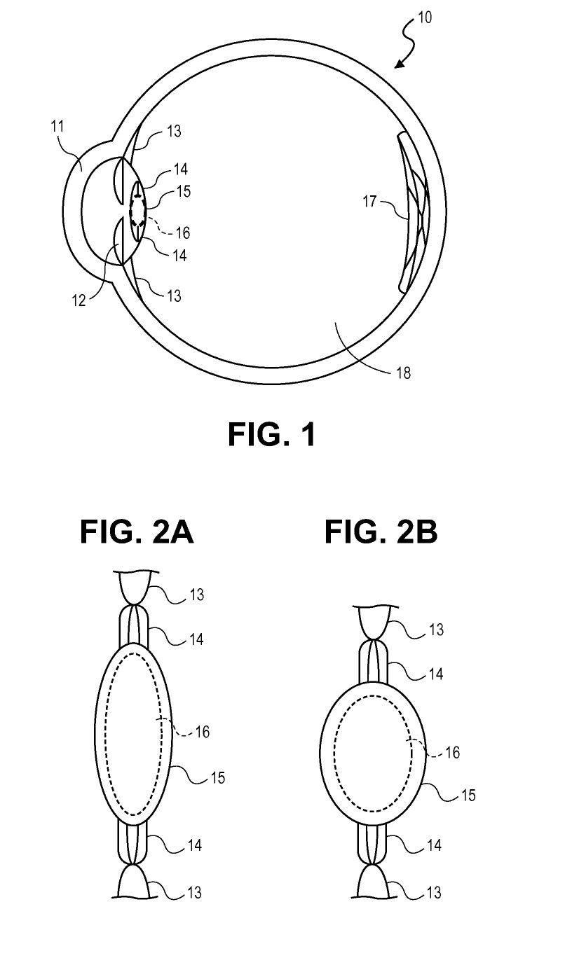

### DETAILED DESCRIPTION OF THE INVENTION

The disclosure relates generally to lenses and methods of accounting for patient variability in lens capsule size, inaccurate measurements of a capsule, and/or changes that can occur in the eye or to the intraocular lens after implanting the intraocular lens in the capsule. Variability in capsule sizes and inaccurate measurements of a capsule can lead to a mismatch in size between the intraocular lens and the capsule. Changes that can occur in the eye after removal of the native crystalline lens followed by implantation of an intraocular lens include changes to the lens capsule. Examples of changes to the lens capsule include, without limitation, capsular contraction (characterized by a fibrotic response), capsular stiffening, growth of the capsule, thickening or thinning of the capsule, any type of capsular healing response, capsular expansion due to healing or a torn or oblong capsularhexis, etc. While capsular contraction is primarily referred to herein, the intraocular lenses can be adapted to account for other types of changes to the capsule after implantation.

While the disclosure herein may primarily refer to an “accommodating intraocular lens” (“AIOL”), the embodiments and methods are not limited to AIOLs, but may also apply to a suitable non-accommodating intraocular lens as well (collectively, “IOL”). “Intraocular lens,” “IOL,” “accommodating intraocular lens,” and “AIOL” as used herein can therefore be referring to a non-accommodating intraocular lens and/or an accommodating intraocular lens. “Lens” as used herein can therefore include both non-accommodating intraocular lenses and accommodating intraocular lenses. Some embodiments, however, specifically describe an accommodating intraocular lens which both accounts for capsule mismatch and/or a capsular response and accommodates in response to ciliary muscle contraction and relaxation.

Before an IOL is implanted within a patient's capsular bag, the capsule is generally measured. Once a capsule, or properties of the capsule (e.g., diameter), is measured, an appropriately sized IOL is selected for implantation. In some embodiments, the appropriate IOL is chosen from a kit of IOLs, each having a different diameter sized for a particular capsule size (as measured or estimated). An alternative for using a kit is to design an IOL with the desired diameter based on the capsule measurement(s). In alternative embodiments (described in more detail below), however, it may not be necessary to measure capsule diameter. Some intraocular lenses described below are adapted such that they automatically account for size mismatch and/or changes that can occur post-implantation without having to measure the capsule diameter. Providing an intraocular lens that can account for these issues without having to measure the capsule diameter would provide a significant advantage by simplifying the overall implantation procedure.

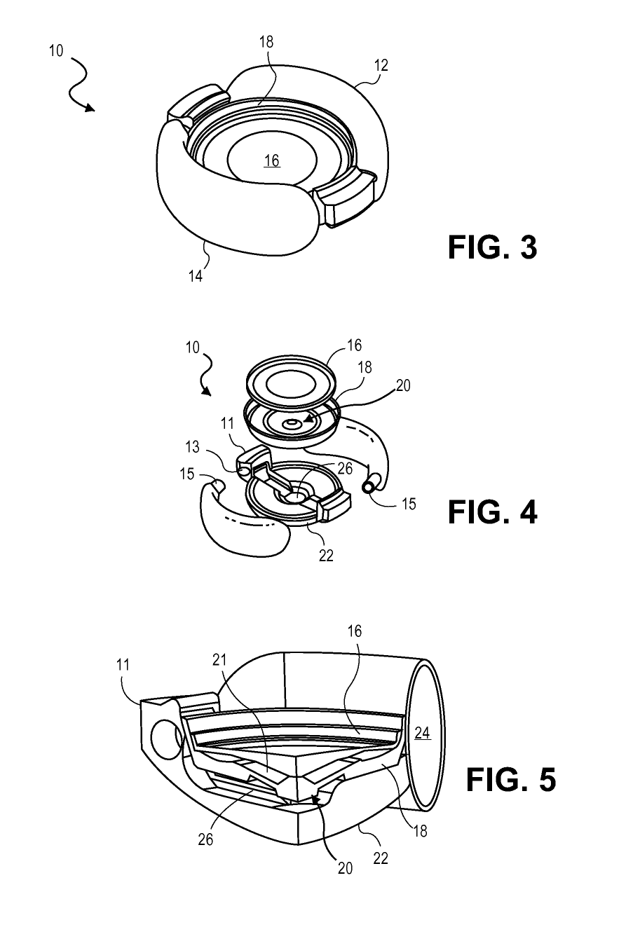

FIGS. 3-5 show a merely exemplary embodiment of accommodating IOL 10, described in more detail in co-pending U.S. patent application Ser. No. 12/177,857, filed Jul. 22, 2008, which is incorporated by reference herein. IOL 10 includes a peripheral non-optic portion comprising haptics 12 and 14. The IOL also includes an optic portion including anterior lens element 16, intermediate layer 18 which comprises actuator 20, and substrate, or posterior element, 22. The inner surface of haptics 12 and 14 define interior volumes 24 which are in fluid communication with active channel 26 defined by posterior element 22 and intermediate layer 18. As shown, actuator 20 is integral with intermediate layer 18. The haptics have a haptic attachment element 15 (which can be stiff or flexible) which is sized and shaped to fit within buttress bore 13. An adhesive layer can be applied to the outer surfaces of the haptic attachment elements and/or the inner surface of the buttress bore to facilitate attachment of the haptics to the optic portion. The IOL includes a first displaceable media such as silicone oil within the haptics and the active channel. The IOL also includes passive chamber 21 that is defined by anterior element 16 and intermediate layer 18. The passive chamber contains a second displaceable media (e.g., a fluid, elastomer, etc.), which may be the same as the media within the haptics and active channel, or it may be a different displaceable media. The active channel and the passive chamber, as shown, are not in fluid communication. In some embodiments both displaceable media are fluids such as a silicone oil.

After AIOL 10 is implanted within a lens capsule (not shown), deformation of haptics 12 and 14 in response to ciliary muscle movement displaces the displaceable media between interior volume 24 and active channel 26. When the displaceable media is displaced into the active channel from the haptics, the pressure in the active channel increases relative to the pressure in the passive chamber, causing actuator 20 to deflect in the anterior direction. This causes the curvature of anterior element 16 to change, thereby increasing the IOL power in this accommodated configuration.

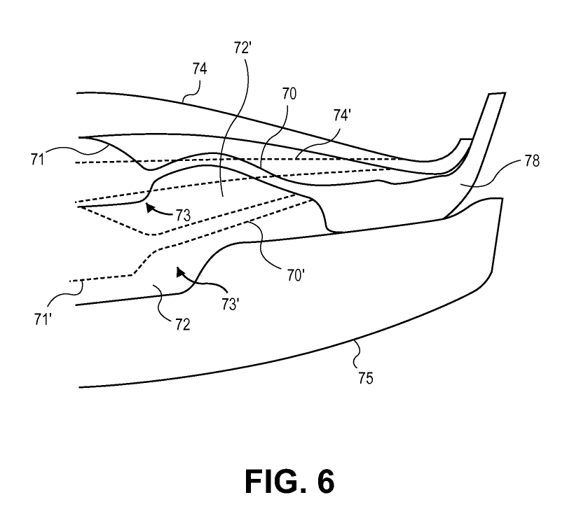

FIG. 6 is a partial cross-sectional view of an optic portion of an exemplary AIOL (haptics not shown) showing roughly half of the optic portion in a disaccommodated state (dashed lines) and an accommodated state (solid lines). The AIOL includes anterior element 74, intermediate layer 78 which includes actuator 73, and posterior element 75. Actuator 73 is comprised of deflection element 71 and bellows 70. When the pressure is increased in active channel 72, bellows 70 change configuration from the generally conical shape of the disaccommodated state to a curvilinear configuration of the accommodated state. Deflection element 71 is forced in the anterior direction due to the increase in pressure in the active channel. This causes anterior element 74 to deflect in the anterior direction as well, steepening the curvature of the anterior element and thereby increasing the power of the lens, as is shown in the accommodated state (solid lines) in FIGS. 6.

In some embodiments, the diameter of the IOL to be implanted can be established by altering the diameter of the optic portion of the lens, the size of the peripheral portion of the IOL, or a combination of the two. For example, the IOL diameter can be varied by varying a dimension of a haptic.

In some embodiments, the appropriateness of the size of the IOL is not dependent (or at least not entirely dependent) on an outer dimension of the IOL. In these embodiments, exemplary alternative aspects of the IOL that can be adjusted include, without limitation, an internal dimension of the IOL or a dimension of specific components of the IOL, the way in which the IOL is manufactured (e.g., the method of bonding various IOL components), the volume of a displaceable media disposed within at least a portion of the IOL. The outer diameter of the IOL can, however, be varied while additionally adjusting a different aspect of the IOL.

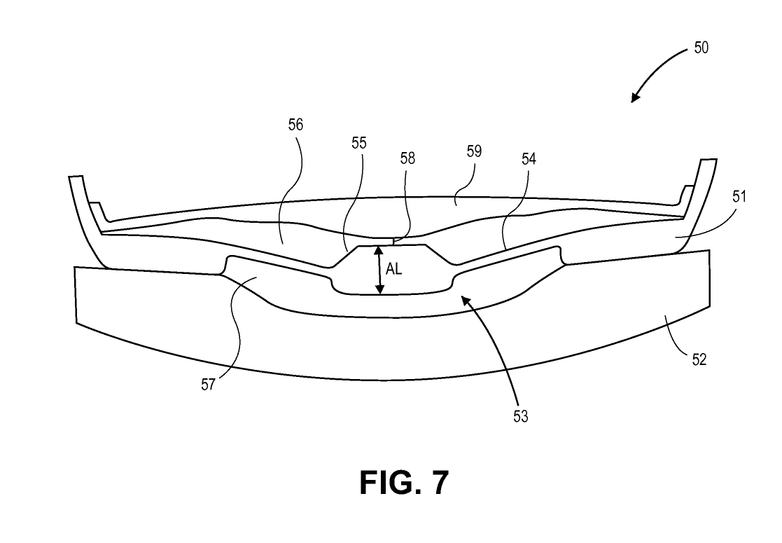

FIG. 7 illustrates an embodiment in which an optic element of the embodiments in FIGS. 3-6 is adjusted to account for mismatch in capsule/lens sizes and/or a change in the eye post-implantation. FIG. 7 illustrates a cross-sectional view of an exemplary embodiment of IOL 50 in a disaccommodative configuration (haptics not shown). One difference between the IOL in FIG. 7 and those in FIGS. 3-6 is that deflection element 55 of actuator 53 is not in contact with anterior element 59 the entire time a flowable media is being displaced towards deflection element 55. In the embodiment in FIG. 7, deflection element 55 is not in contact with anterior element 59 as the fluid pressure in the active channel initially begins to change. FIG. 7 can represent a disaccommodative configuration, in which case deflection element 55 is not in contact with anterior element 59 when the IOL is in the disaccommodative configuration. The deflection element in the IOL shown in FIGS. 3-6 is at least in contact with the anterior element once the lens is implanted, and may additionally be bonded thereto. The IOL in FIG. 7 is shown with dead zone 58 defined by the distance between the deflection element 55 and the anterior element 59. In this embodiment, the aspect of the IOL that is varied to provide an appropriately sized IOL for implantation is the length of the dead zone. Other internal dimension of the lens can also be adjusted.

In the embodiment in FIG. 7 and others described herein, the power of the AIOL changes in a non-linear manner in response to capsular forces on the AIOL. That is, the slope of a hypothetical power vs. capsular forces plot for the AIOL is not constant throughout the entire range of capsular forces on the AIOL. In general, the slope of the hypothetical plot increases with increasing capsular forces. The capsular forces can be due to an accommodative response, size mismatch, changes in the capsule post-implantation, etc. The AIOL can have any type of non-linear response. For example, the response may be considered to occur in a plurality of discrete stages. Each of the discrete stages can have a uniform power change in response to increasing capsular forces. In some embodiments the discrete stages do not, however, have uniform power changes. The response of some intraocular lenses does not occur in a plurality of discrete stages, but rather the power change can be considered to be continuous with increasing capsular forces. Some responses may be considered to be a combination of a continuously increasing power which incorporates one or more discrete stages. All other types of non-linear responses are also included herein.

In some embodiments the AIOL undergoes a first power change phase in response to capsular reshaping and a second power change phase in response to additional capsular reshaping, wherein the power change during the first phase is different than the power change during the second phase. The term “phase” as used herein is not meant to indicate a discrete step in the overall response of the AIOL. “Phase,” as used herein refers generally to a portion of the AIOL's non-linear response, and can also include the entire non-linear response. In general, a phase, or portion of the response (which can be arbitrarily determined), is associated with a change in power of the AIOL. That is, the phase power change is the difference in power between the end of the phase and the beginning of the phase. In general, the change in power of the IOL during a first phase of the non-linear response is less than the power change during at least a second phase of the non-linear response. That is, the slope of the power change is not constant, but increases at at least some point during the non-linear response. In some embodiments, the power change during a first phase is substantially zero, such that there is substantially no change in IOL power during the first phase. In other embodiments the power change during a first phase is not substantially zero, but it is less than the power change during a second phase. The power change during a first phase may be substantially less than the power change during a second phase.

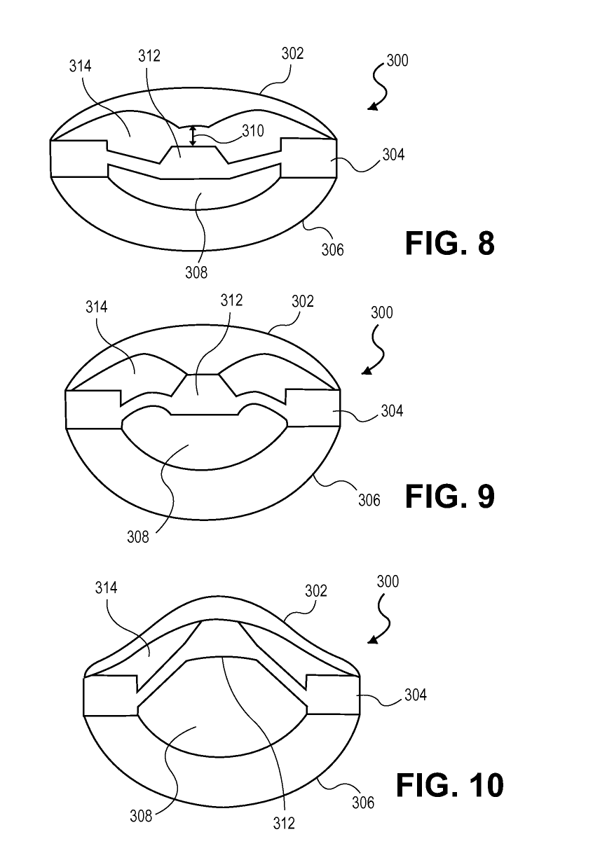

FIGS. 8-10 show cross-sectional side views of configuration changes of an optic portion of an exemplary IOL (haptics not shown) with a dead zone. FIG. 8 shows the IOL in an initial configuration after manufacture and theoretically immediately after implantation in the capsule. FIG. 10 shows the IOL in a fully accommodated configuration, and FIG. 9 shows a configuration in which the actuator is in contact with the anterior element. IOL 300 includes anterior element 302, intermediate layer 304 (including deflection element 312 of the actuator), and posterior element 306. The deflection element 312 and anterior element 302 define dead zone, or gap, 310.

When there is little or no pressure in active channel 308 or in passive chamber 314, the geometry and passive fluid state is such that dead zone 310 exists between deflection element 312 and anterior element 302. As described above with respect to FIG. 3-6, an increase in the fluid pressure in active channel 308 (due to the displacement of media from the haptics towards active channel 308) causes deflection element 312 to deform in the anterior direction (similar to the deformation shown in FIG. 6). Because deflection element 312 is not in direct contact with anterior element 302 (i.e., because there is a dead zone 58 between the two), however, force is not initially transferred directly from deflection element 312 to anterior element 302. The amount of force transferred to the anterior element, for a given initial increase in pressure in the active channel, will therefore be greater in the embodiments shown in FIGS. 3-6 than in the embodiments shown in FIG. 7-11 (assuming the respective AIOLs are manufactured in the same manner in all other respects). The dead zone allows the AIOL to change configurations in response to capsular forces such that the power change in response to increasing capsular forces is non-linear. The configuration change in this embodiment is the deformation of deflection element 312. In this embodiment, the optic portion of the AIOL changes configuration in response to capsular forces acting on the AIOL.

As the pressure in active channel 308 continues to increase, deflection element 312 continues to deflect in the anterior direction and contacts anterior element 302, as in the configuration shown in FIG. 9. As the pressure in active channel 308 continues to increase, deflection element 312 continues to deflect in the anterior direction and applies (and continues to apply as long as the deflection element continues to deflect) a direct force to a portion of anterior element 302, causing anterior element 302 to deflect in the anterior direction. This changes the curvature of anterior element 302, thereby increasing the optical power of the IOL.

In general, the change in optical power of the lens is greater between FIGS. 9 and 10 than between FIGS. 8 and 9. That is, the power of the lens changes more after the deflection element contacts the anterior element than before the deflection element contacts the anterior element. This occurs because the change in curvature of the anterior element is substantially greater between FIGS. 9 and 10 than between FIGS. 8 and 9. The dead zone is one way in which the exemplary lens can be adapted such that the power response of the AIOL to capsular reshaping is not linear. In the embodiment in FIGS. 8-10, the optical power change of the lens is greater after the deflection element contacts the anterior element.

There may be a physiological advantage to having at least some power increase in response to initial capsular forces. For example, it may be advantageous to alert the brain that the accommodative effort is beginning to bring about the desired change in power.

In use, after the capsule is measured, AIOL 300 can be selected so that after implantation, dead zone 310 will account for capsular contraction around AIOL 300 and/or a mismatch in size between AIOL 300 and the capsule. The capsular contraction and/or mismatch in size can therefore cause the AIOL to change configurations to that shown in FIG. 9. The power change of the AIOL is non-linear such that a permanent myopic shift is prevented or at least minimized. AIOL 300 can also undergo accommodation in response to ciliary muscle movement to the fully accommodated configuration shown in FIG. 10.

In some embodiments the anterior element is generally spheric in the disaccommodated configuration (FIG. 8) and becomes aspheric as soon as the pressure in the active channel begins to increase. In these embodiments, in reference to FIGS. 8-10, the anterior element becomes aspheric as soon as the lens begins to transition from the configuration shown in FIG. 8 towards the configuration in FIG. 9. The anterior element, however, is less aspheric in the configuration in FIG. 9 than FIG. 10. Also, the rate of change in curvature of the anterior element is less between FIGS. 8 and 9 than between FIGS. 9 and 10. The power of the lens therefore increases as the lens changes configuration between FIGS. 8 and 9. This change is relatively insignificant, however, when compared to the overall power change between the configurations in FIG. 8 and FIG. 10.

In some embodiments, however, the anterior surface is spheric in the disaccommodated configuration and remains spheric (or substantially spheric) until the deflection element contacts the anterior element. In these embodiments, referring to FIGS. 8-10, the anterior element is at least generally spheric in FIGS. 8 and 9, and become aspheric as the lens changes configuration from FIGS. 9 to 10. After contact is made between the deflection element and the anterior element, the anterior element becomes aspheric as the anterior element is deflected in the anterior direction.

In some embodiments, as the pressure in the active channel continues to increase (either before or after the deflection element contacts the anterior element), the actuator continues to deflect in the anterior direction. Because of the size of the deflection element relative to the anterior element, the fluid in passive chamber 314 redistributes and creates an aspheric effect in the anterior element. This further increases the power of the IOL for a smaller aperture.

In embodiments in FIGS. 8-10 in which the power changes from the configuration in FIG. 8 to FIG. 9, the AIOL provides for a lower power change rate as initial forces are applied to the IOL (FIG. 9), and a higher power change rate as additional forces continue to be applied (FIG. 10).

The embodiments of the IOLs described herein that do not have a dead zone or other feature to accomplish similar goal(s) change power in a more linear fashion in response to capsular reshaping than the IOLs with a dead zone (assuming all other aspects of the IOLs are the same and the capsular bags are of the same size). The use of a dead zone allows for initial forces to be applied to the IOL from the capsular bag while minimizing the amount of myopic shift that occurs in the patient.

In use, after the AIOL is implanted, capsular contraction and any mismatch in lens/capsule size can change the configuration of the AIOL. In some instances, even after capsular contraction and mismatch in size have been accounted for, the disaccommodated configuration of the AIOL may still have a dead zone (see FIG. 8). That is, the capsular forces applied to the AIOL do not fully reconfigure the AIOL to the configuration shown in FIG. 9. In these embodiments, the use of a dead zone (or other similarly-acting features) can be thought of as creating a plurality of types, or phases, of accommodation. For example, rather than a generally linear accommodative response from ciliary muscle movement, the response is non-linear. This amounts to varying power rates of change in the lens. For example, as described above with respect to FIGS. 7-10, initial forces applied to the capsular bag result in deformation of the deflection element but yield relatively little or substantially no accommodation (i.e., relatively little or substantially no optical change in the lens). This can be considered a first type, or phase, of accommodation. After the deflection element contacts the anterior element, however, the accommodative response increases (i.e., the rate increases). This can be considered a second type, or second phase, of accommodation. Additionally, one or more transition, or intermediate, levels of accommodation can exist between the two types of accommodation. That is, the transition period that occurs just as the deflection element begins to contact the anterior element can result in more accommodation that the first type of accommodation, but less than the second type of accommodation. There can any number of types, or phases, of accommodation with the use of a dead zone, and the above example is merely exemplary.

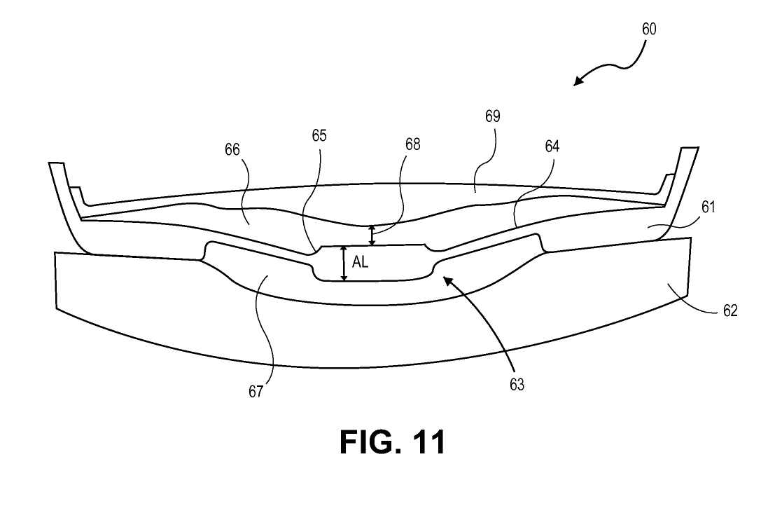

Adjusting the length of the dead zone can control the power rate change in the IOL in response to a given amount of capsular force. By way of illustration, IOL 50 in FIG. 7 has a smaller dead zone 58 than the dead zone 68 in IOL 60 shown in FIG. 11. When IOL 50 and IOL 60 are placed into capsular bags of exactly the same size (and all other aspects of the IOLs being equal), forces applied from the capsular bag to the haptics will cause deflection element 55 to contact anterior element 59 sooner than deflection element 65 will contact anterior element 69. This will cause anterior element 59 to ultimately deflect more than anterior element 69. The change in optical power of IOL 50 from a disaccommodated configuration to an accommodated configuration will therefore be greater than the change in optical power of IOL 60. The power rate of change is also greater in IOL 50 than in IOL 60.

Alternatively, the difference in diopter power of IOL 50 and IOL 60 between their respective disaccommodated and accommodated states may be substantially the same. For example, IOL 60 can be configured such that there is a delay in the deflection of anterior element, but once the deflection element contacts the anterior element, the power rate change for IOL 60 is greater than the power rate change in IOL 50, with the result that the anterior elements are ultimately deflected the same amount.

As described above, capsule sizes can vary from patient-to-patient, and even from eye-to-eye. If an IOL is implanted which is too large for the capsule, the capsule can apply permanent forces to the haptics, which can increase the pressure in the active channel and increase the power of the lens. The patient can therefore develop a permanent myopic shift. Alternatively, the IOL may be too small, resulting in insufficient or inefficient accommodation. To account for this, an IOL with a desired dead zone, based on the measured capsule size, can be implanted into the capsule. For example, if a capsule is measured and has a relatively small diameter such as, for example, about 9.7 mm, the capsule will apply forces to the IOL upon implantation which can result in a myopic shift. To account for this, an IOL can be selected with a relatively large dead zone, such as the IOL shown in FIG. 11. A larger dead zone will allow more force to be applied to the IOL from the capsular bag and yet produce relatively little (compared to a subsequent period of accommodation) or substantially no accommodation. Upon implantation, the small capsule will likely apply force to the IOL even absent ciliary muscle contraction, but the dead zone will allow the actuator to deform without contacting the anterior element. This prevents direct force from being applied from the actuator to the anterior element, yielding relatively little or substantially no power change. A myopic shift is therefore prevented or at least reduced. The AIOL can also accommodate in response to ciliary muscle movement as described above.

Alternatively, if the capsule is measured and has a relatively large diameter of about, for example 11.3 mm, the outer diameter of the IOL may not be large enough to provide a good fit between the IOL and the capsular bag, and the capsule bag may change configuration in response to ciliary muscle contraction without causing a sufficient optical power change in the IOL. To account for this, an IOL with a smaller (or even nonexistent) dead zone can be chosen, such as the IOL 50 shown in FIG. 7. Once implanted in a relatively large capsule, forces transferred to the IOL from the capsule bag will more quickly (and at a greater rate) change the optical power due to the shorter dead zone. Stated alternatively, force applied to the IOL from the capsule will result in a more efficient deflection of the anterior element than an IOL with a greater dead zone, if placed in the same capsular bag.

In use, because it is very difficult to obtain capsular bag measurement that are accurate to greater than about +/−300 microns, and because the capsule diameter can vary from small to large sizes by about 1.5 mm, a risk may always exist that an IOL will be too large for the capsule, and that a large, permanent myopic shift will result. To account for such a risk, dead zones as described herein can be used. By way of example, a capsular bag capable of applying 10 units of contractile force can, theoretically, linearly produce 10 diopters of accommodation. While this may be ideal, the risk of a myopic shift may always exist. It may therefore be safer to make the IOL's response to capsular forces non-linear. For example, the IOL can be designed such that the first 4 units of force produce little or no accommodation, while the next 6 units produce the full 10 diopters of accommodation. In this example the IOL is designed with a 4-unit dead zone. If the IOL is too large for the capsule and the capsule therefore exerts a permanent force on the IOL, the power of the lens will not shift, or will shift relatively little, until the force on the IOL exceeds 4 units. By ensuring that the dead zone is large enough to account for any permanent force due to a size mismatch between the IOL and the capsular bag, a myopic shift can be prevented or at least minimized.

Alternatively, a capsular bag can be thought of as providing 10 units of dimensional change (as opposed to 10 units of force), which can theoretically produce 10 diopters of accommodation. Similar to the example given above, 4 units of dimensional change can be factored in for size mismatch and/or capsular contraction. In the example, the force applied by the capsule may not matter as much as the dimensions involved.

Selecting a lens with a non-linear power shift response as described above can also be used to adapt to capsular bag contraction which can occur after implanting the lens. Capsules frequently naturally respond by contracting and shrinking around the IOL, creating a permanent force on the lens as described above. Upon contraction, the capsular bag reshaping can cause a change in the power of the IOL, resulting in a permanent myopic shift in the eye (even when the ciliary muscles are not contracting). Incorporating a dead zone into the lens which provides for relatively little or substantially no accommodating in response to capsular forces allows the capsule to undergo this natural healing process while minimizing or maybe even avoiding a permanent myopic shift.

In some embodiments a kit of lenses is used, each lens with a different dead zone length. The capsule is initially measured, and based on the measurement a specific lens is chosen. One additional advantage of varying the dead zone is that an outer dimension of the lens need not be adjusted. Alternatively, however, the kit can include lenses with varying outer dimensions (e.g., outer diameter), and for a given outer dimension size, the kit can include lenses with varying dead zones. This can provide even more options for choosing the most appropriately sized IOL.

In some embodiments an IOL with a first dead zone can be used if the capsule size is measured to be below a predetermined low threshold level, while an IOL without a dead zone (or an IOL with a second dead zone smaller than the first dead zone) can be used if the capsule size is measured to be above a predetermined high threshold level. It may be desirable to use an IOL without a dead zone when there is very little, or no, risk that the IOL will be too large for the capsule.

There are various ways to modify the length of the dead zone in the exemplary IOLs described herein. One way to adjust the dead zone is to adjust the axial length (along the optical path of the lens) of the deflection element. For example, the deflection element 58 in the embodiment in FIG. 7 has a longer axial length “AL” than the axial length “AL” of the deflection element 65 in the embodiment in FIG. 11. In some embodiments the axial length of the dead zone is between about 0 microns and 400 microns. The deflection element can be a polymer cured in a mold to have a specific axial length, or alternatively the deflection element can be machined down after curing to a smaller axial length.

An alternative method of varying the dead zone is to adjust the volume of displaceable media in the passive chamber. Increasing the volume of displaceable media in the passive chamber increases the dead zone. This occurs because increasing the amount of passive displaceable media increases the posteriorly directed force to the actuator and/or the anteriorly directed force to the anterior element, thereby increasing the distance between the actuator and the anterior element. Similarly, decreasing the volume of passive displaceable media decreases the dead zone.

Similarly, the volume of fluid in the active channel can be adjusted to adjust the dead zone.

The dead zone can also be adjusted by varying the thickness (i.e. axial length) of the anterior element. Decreasing the axial length of the anterior element increases the dead zone, whereas increasing the axial length of the anterior element decreases the dead zone. The dead zone can also be adjusted by varying any of the IOL elements described herein.

In the embodiments above, a portion of the optic portion of the IOL undergoes a configuration change in response to capsular forces. As is described below, features alternative to the dead zone (or in addition to) can be incorporated into the IOL to provide or assist in providing the system with the ability to deform or change configuration during a first portion of the non-linear response.

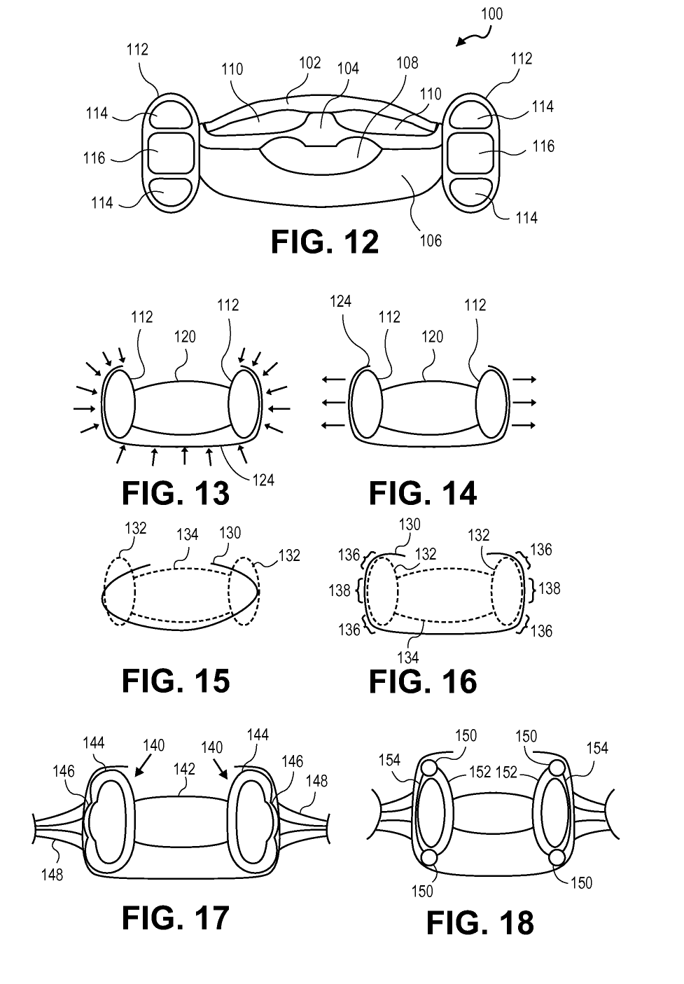

FIG. 12 illustrates a variation of an intraocular lens which accounts for variability of capsule size and/or a change in the eye or intraocular lens post-implantation, yet accommodates in response to ciliary muscle movement. Intraocular lens 100 includes an optic portion including anterior element 102, intermediate element 104, and posterior element 106. Anterior element 102 and intermediate element define passive chamber 110, while intermediate element 104 and posterior element 106 define active channel 108. Haptics 112 include active chamber 116 and passive chambers 114. Passive chamber 110 houses a first displaceable media, such as a liquid, and is in communication with passive chambers 114, while active channel 108 and active chambers 116 are in communication and house a second displaceable media. In FIG. 12 intermediate layer is shown to be in contact with anterior element 102, but the lens may also be manufactured to have a gap between anterior element 102 and intermediate element (as in the embodiments above).

After intraocular lens 100 is positioned with capsule 124 (see FIG. 13), the capsule may undergo a healing response, contracting around the implanted intraocular lens and applying forces on the intraocular lens in the directions of the arrows shown in FIG. 13. The capsule may alternatively, or in addition to, be smaller than determined from a capsule size determination (i.e., there is a mismatch in size between the intraocular lens and the capsule). Based on the mismatch in size, the capsule can apply similar forces on the intraocular lens. When the capsule applies forces to the intraocular lens as shown in FIG. 13, the communications between active chamber 116/active channel 108 and passive chambers 114/passive chamber 110 keeps the pressures within active channel 108 and passive chamber 110 substantially equal (or at least minimizes the difference between the pressures). Because the optical power of the intraocular lens generally changes when the pressure in active channel 108 increases relative to the pressure in passive chamber 110, keeping the pressures substantially the same allows the intraocular lens to take into account any capsular contraction and/or mismatch in size between the patient's capsule and intraocular lens without yielding an optical power change (or at least minimizing any power change). That is, the lens will remain substantially in a disaccommodated configuration when capsular forces due to capsular contraction and/or mismatch in sizes occur.

Intraocular lens 100 also allows for accommodation to occur during ciliary muscle movement. Zonules extend generally radially from the capsule (see FIGS. 2A and 2B), and zonular forces on the capsule during ciliary muscle relaxation are shown generally in FIG. 14. During ciliary muscle contraction, the tension in the zonules decreases. The radial extension of the zonules allows the capsule to compress the haptics in a radial direction, which creates a greater pressure change in the active channel 116/108 than in passive chamber 112/110, which is relatively unaffected by radial compression. The increase in active channel pressure relative to passive chamber 110 causes the intermediate element 104 to deform as described above. The change in configuration of the intermediate 104 causes the curvature of the anterior element 102 to change, thereby changing the power of the lens. Intraocular lens 100 illustrates one exemplary manner of isolating the radial zonular movement associated with ciliary muscle movement from other capsule forces associated with capsule size mismatch or capsular contraction following implantation.

FIGS. 15 and 16 illustrate a variation of an intraocular lens which can be considered to be a one-size lens which can be considered to fit all different capsule sizes (or substantially all) as well as to account for capsular forces not caused by ciliary muscle movement. In this and similar embodiments, haptics are designed to stretch the capsule radially. FIG. 15 illustrates the relative sizes of an intraocular lens to be implanted with native capsule 130, while FIG. 16 shows the intraocular lens implanted within capsule 130, stretching the capsule 130 to accommodate the size of the haptics. In this embodiment, haptics 132 have regions 136 which are stiffer than the capsule. In this embodiment, each of haptics 132 has an anterior and a posterior region 136 which is stiffer than the capsule. Each haptic also includes equatorial region 138 which are less stiff than regions 136, and in some embodiments are about as stiff as the capsule.

The portions of the haptics which are stiffer than the capsule are configured to stretch all (or substantially all) capsules, regardless of their size. The intraocular lens is therefore relatively independent of the capsule size of the patient, as all capsules will be stretched once the lens is implanted. Equatorial regions 138, however, which are less stiff than regions 136, allow for the power of the lens to be adjusted during ciliary muscle movement when the zonular forces on the capsule change. Because the zonules are light springs, the ciliary muscles may be able to produce pressure changes in an intraocular lens even if the intraocular lens is larger or smaller than the native crystalline lens. This embodiment provides an intraocular lens which is essentially insensitive to capsule size yet is highly sensitive to ciliary muscle movement.

In some embodiments the haptics are rigid in the non-zonular contact regions and are compliant in the zonular contact zones. FIG. 17 illustrates a variation of an intraocular lens in which haptics 140 include a first region 144 which is stiff compared to the capsule. The stiffness of first region 144 can be controlled through, for example without limitation, wall thickness, wall geometry, material selection, etc. First region 144 is disposed within the capsule such that is positioned in a non-zonular contact zone (or substantially in a non-zonular contact zone). Second region 146 of haptics 140 are more resilient that first regions 144, and in FIG. 17 second region 146 is made more resilient by having a thickness less than the thickness of first region 144. Second regions 144 are in zonular contact zones, and thus are made more resilient to respond to zonular tensioning during ciliary muscle movement. The resiliency can be adjusted by, for example without limitation, wall thickness, wall geometry, material selection, etc.

FIG. 18 illustrates a variation in which the haptics include stiff solid rings 150 which are stiffer than the capsule, first regions 152 which are stiffer than the capsule, and second regions 154 which are less stiff than first regions 152. Second regions 154 are generally disposed in zonular contact zones within the capsule, while rings 150 and first regions 152 are generally disposed in non-zonular contact zones. Stiff solid rings 150 can be entirely separate from the fluid filled haptics defined by regions 152 and 154. Stiff solid rings could be made from another material such as, for example without limitation, PMMA, Titanium, Nickle Titanium, etc.

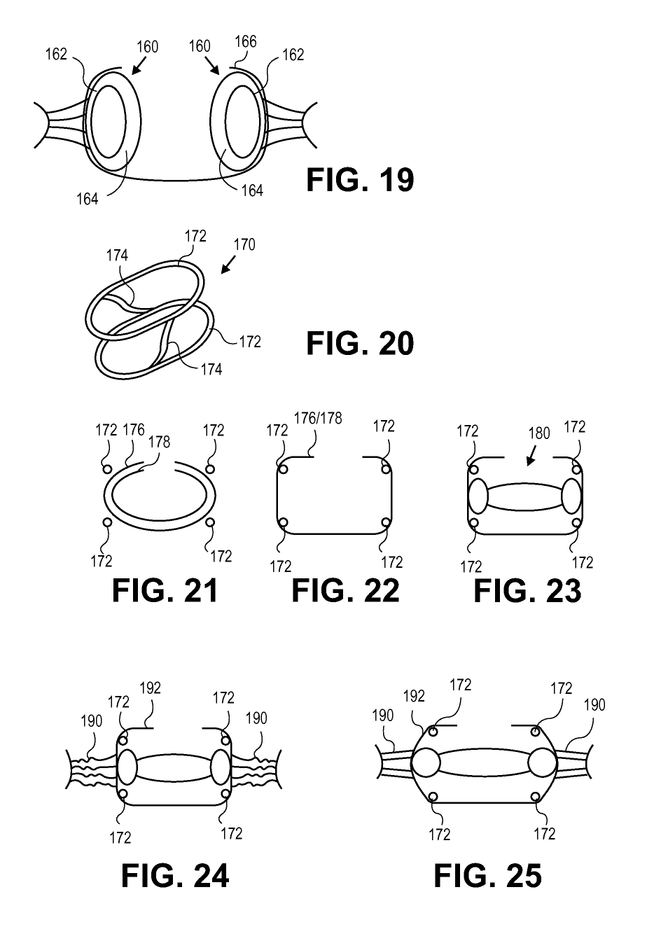

FIG. 19 illustrates an alternative variation in which haptics 160 include first region 164 which is disposed in a non-zonular contact zone and second region 162 which is disposed in a zonular contact zone within capsule 166. In this embodiment second region 162 has a smaller wall thickness than first region 164. The stiffness of the regions of the haptics can be controlled in other ways as well, such as those described herein.

Other merely exemplary features which can be used to stretch out the capsule along the optical axis (as in the embodiments above) while maintaining radial compliance include, for example, I-beams, rings that utilize hoop forces, etc.

FIGS. 20-22 illustrate a variation in which the intraocular implant includes capsular tension frame 170. Frame 170 includes annular elements 172 and support members 174. The annular elements are stiffer than the capsule and can be made from, for example without limitation, PMMA, Nitinol, etc. Support members 174 (two are shown but one or more may be used) maintain annular elements 172 at a fixed distance apart from one another. Support members 174 are also relatively stiff, and can be made from the same or different material as annular elements 172. Frame 170 is collapsible and can be inserted through a delivery device via an incision in the eye. If necessary, the geometry may be adjusted to ease insertion. For example, the annular elements 172 can be split so they can assume an elongated delivery configuration for insertion.

Frame 170 is first positioned within the capsular bag. Frame 170 can be sized such that a single-sized frame will stretch all types of capsules. For example, the frame can be sized such that all patients with lens capsules from about 9 mm to about 10.5 mm will stretch over the frame. Because the frame is stiff relative to the capsule, the geometry of the frame/capsule system will be dominated by and therefore dictated by the frame geometry. All patients who have the single-sized frame inserted into their capsule will have a capsule that is essentially the size of the frame, rather than the about 9 mm to about 10.5 mm size before frame insertion. FIG. 21 illustrates a single-sized frame with annular elements 172 relative to two different capsules 176 and 178. Capsule 176 is larger than capsule 178. FIG. 22 illustrates the size of both capsules 176 and 178 after the frame is positioned within the capsules. Both capsules now have substantially the same size and configuration.

After the capsule is stretched by capsular tension frame 170, intraocular lens 180 is then positioned within the capsule, as shown in FIG. 23. The lens/capsule interface (which in FIG. 23 is the capsule/haptic interface) can be arranged such that it does not engage support members 174. The zonules, which act like springs, should remain in tension even if the capsule is on the large end of the capsule size spectrum.

FIG. 25 illustrates zonules 190 pulling capsule 192 to a diameter larger than the diameter of capsular tension frame 170 (annular elements 172 shown) as the eye attempts to disaccommodate. When the eye attempts to accommodate (or if the capsule contracts after implantation), as shown in FIG. 24, the capsular tension frame prevents the haptics and the lens from being crushed or activated beyond the diameter dictated by the frame. The capsular tension frame therefore can act as an inner haptic stop which has the property of limiting inward motion of the capsule onto the lens without significantly changing the ability of the equator of the capsule to perform work on the lens or other intercapsular device.

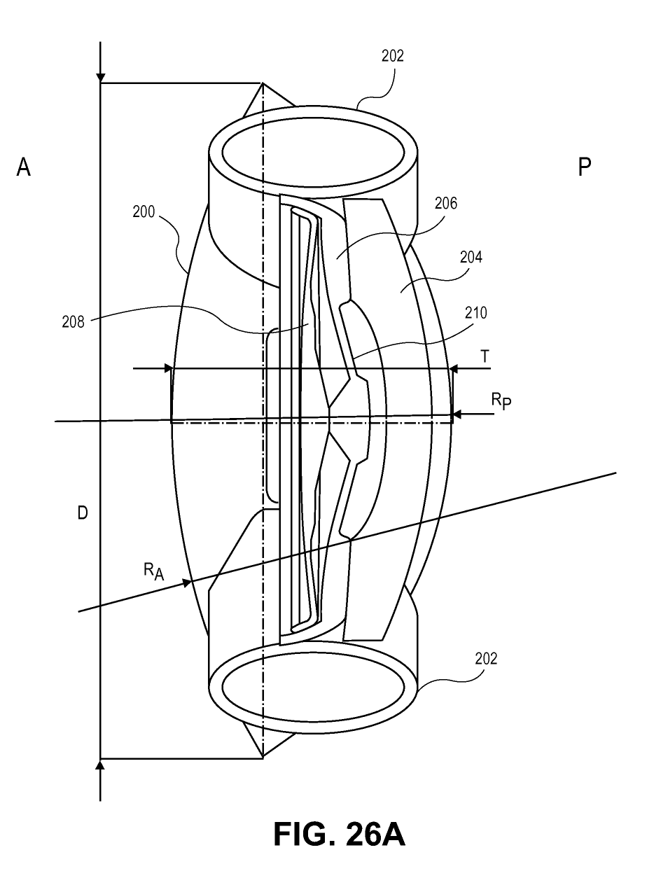

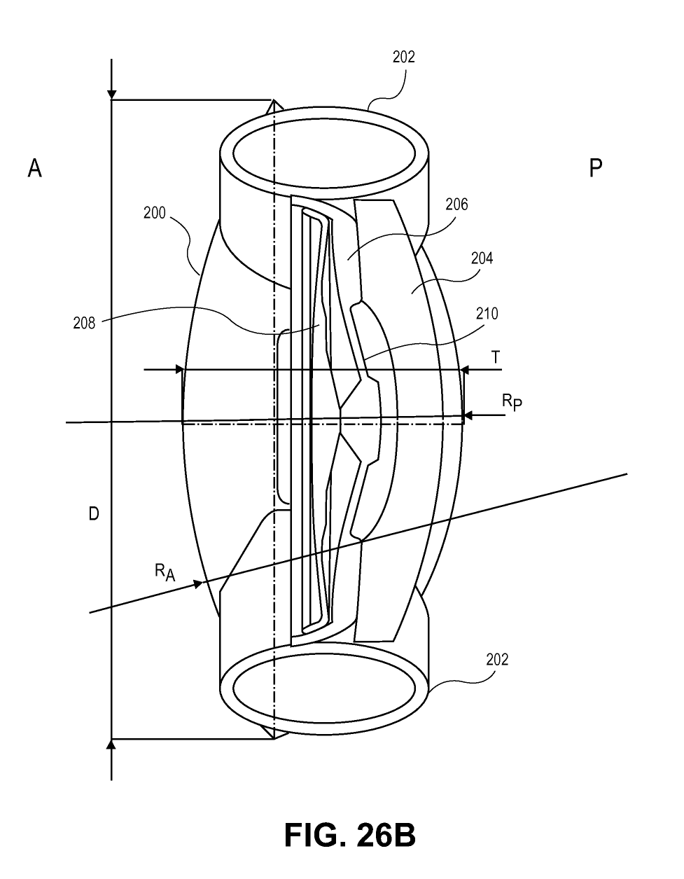

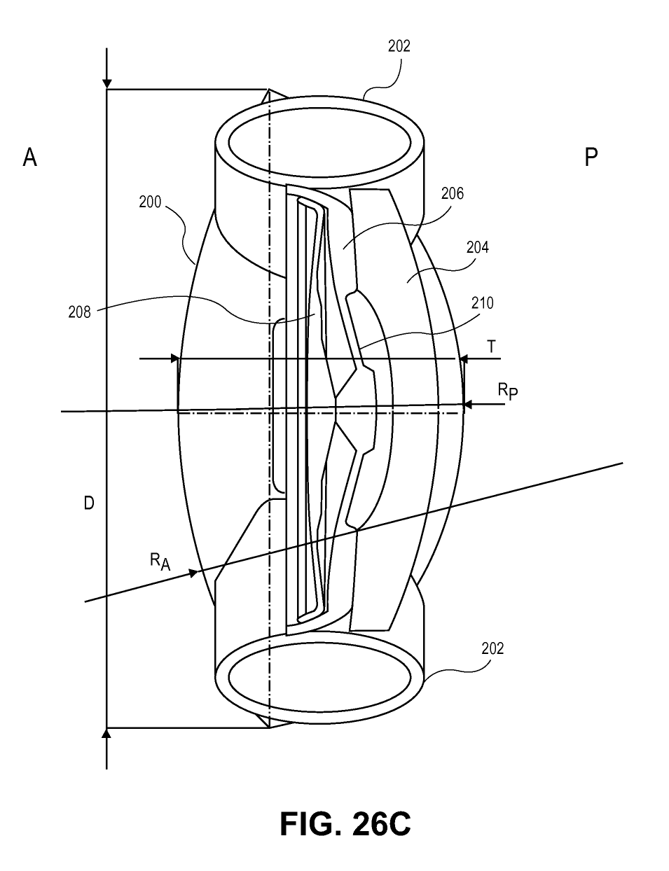

FIGS. 26A-26C, respectively, illustrate cross-sectional views of an alternative embodiment of an AIOL superimposed on illustrations of cross-sections of three different capsular bags. FIGS. 26A-26C show the relative sizes of the cross-section of the AIOL and the capsular bags. In the Figures, the anterior direction “A” is generally towards the left on the pages (towards the cornea), and posterior direction “P” is generally towards the right on the pages (towards the retina). The intraocular lens includes a peripheral portion including haptics 202, and an optic portion comprising posterior element 204, intermediate element 206, and anterior element 208. The intraocular lens accommodates in response to ciliary muscle movement as described in the embodiment shown in FIGS. 3-6.

Haptics 202 are shaped and sized such that they are larger than the peripheral portions of the capsule in which they are to be positioned. Specifically, in this embodiment, an anterior portion of the haptics extends further in the anterior direction than the capsule, and a posterior portion of the haptics extend further in the posterior direction than the capsule. As described above with respect to the embodiment in FIGS. 3-6, haptics 202, which comprise a flexible material, define an internal chamber in fluid communication with active channel 210. The flowable media (such as a fluid) disposed within haptics 202 and active channel 210 is displaced in response to capsular reshaping due to the interaction between the flexible haptics and the capsule. Due to the relative sizes of the haptics and the capsule, when the intraocular lens shown in FIGS. 26A-26C is implanted in the any of the capsules shown in FIGS. 26A-26C, the peripheral portions of the capsule are reconfigured to accept the haptics, and the capsule therefore applies forces on haptics 202 in the regions where the capsule engages the haptics. Additional forces may additionally be applied to the haptics due to capsular changes post-implantation (e.g., capsular contraction).

The engagement between the haptics and capsule, as well as the size and shape of haptics 202 and capsule 200, provide for substantially no net fluid displacement between the haptics and the active channel when forces are applied to the haptics due to both the mismatch in size between haptics 202 and capsule 200 and changes to the capsule post-implantation. While forces are applied to the haptics, the forces are substantially canceled out, resulting in substantially no net fluid displacement. Providing substantially no net fluid movement results in substantially no pressure increase in active channel 210. As discussed above with respect to FIGS. 3-6, anterior element 208 therefore undergoes substantially no change in curvature, resulting in substantially no power change. The size and shape of the haptics therefore minimize the power change of the intraocular lens, and therefore minimize myopic shift. The intraocular lens still functions as an accommodative intraocular lens in response to ciliary muscle movement, as is described above with respect to FIGS. 3-6.

In the embodiment shown in FIGS. 26A-26C, forces exerted on the superior and inferior portions of the haptics (i.e., the top and bottom in the Figures) will tend to increase the volume of the haptics, cause fluid to be displaced towards the haptics, and therefore tend to decrease the power of the lens. Forces exerted on the anterior and posterior portions of the haptics (i.e., the sides in the Figures), however, will tend to reduce the volume of the haptics, cause fluid to be displaced towards the optic, and therefore tend to increase the power of the lens. The haptics are designed such that the forces that are applied to the top and bottom of the haptics are substantially equal to the forces applied to the sides of the haptics. As a result, there is substantially no change in the volume of the haptics and therefore substantially no fluid movement. In this embodiment, the haptic is designed such that forces applied by the capsule on the haptic produce substantially no change in volume of the haptic and therefore substantially prevent the power of the intraocular lens from changing. The principle can also be applied to non-fluid driven accommodating intraocular lenses that have peripheral regions that deform in response to capsular forces. Depending on the manner in which the power of the lens is configured to change in response to a mismatch in size of a change in the capsule post-implantation, the deformable peripheral region of the lens can be configured such that the net force applied on the peripheral region results in substantially no power change.

Haptics 202 have a generally oval or elliptical cross-section as shown in FIGS. 26A-C. In some embodiments the haptic anterior-to-posterior thickness is about 3.2 mm, the width of the haptics is about 1.2 mm, and the wall thickness of the haptics is about 0.2 mm. The dimensions listed are not intended to be limiting in any way. In some embodiments the diameter of the intraocular lens when compressed by the capsule but not yet moving fluid (i.e., in a disaccommodated configuration) is about 9.1 mm.

In FIG. 26A, capsule 200 has a diameter “D” of 9.80 mm, a thickness “T” of 4.25 mm, an anterior radius of curvature “R_{A}” of 10 mm, and a posterior radius of curvature “R_{P}” of 5.53 mm. In FIG. 26B the capsule has a diameter of 9.8 mm, a thickness of 4.25 mm, an anterior radius of curvature of 7.86 mm, and a posterior radius of curvature of 6 mm. In FIG. 26C the capsule has a diameter of 10.21 mm, a thickness of 4.25 mm, an anterior radius of curvature of 10 mm, and a posterior radius of curvature of 6 mm.

In an alternative embodiment, a dead zone as described above can be incorporated into the AIOL shown in FIGS. 26A-C. Even though haptics 202 provide for substantially no power shift due to capsular reshaping (with the exception of capsular reshaping due to ciliary muscle movement), a dead zone can be incorporated in case there is a minimal amount of fluid displacement towards the deflection element in response to a mismatch in capsule/haptic size and/or a capsular change post-implantation. A dead zone can be incorporated for added assurance that there will be substantially no power shift. Alternatively or in addition to, a dead zone can be incorporated into the lens shown in FIGS. 26A-C to provide for two phases of power change, as is described in more detail above.

Exemplary alternative AIOLs which can be modified to include a dead zone or other features which account for capsular contraction or variability in capsule size can be found in the embodiments in U.S. Pat. No. 7,122,053, U.S. Pat. No. 7,261,737, U.S. Pat. No. 7,247,168, U.S. Pat. No. 7,217,288, U.S. Pat. No. 6,935,743, U.S. Patent Application Publication 2007/0203578, U.S. Patent Application Publication 2007/0106377, U.S. Patent Application Publication 2005/0149183, U.S. Patent Application Publication 2007/0088433, U.S. Patent Application Publication, and U.S. application Ser. No. 12/177,857, filed Jul. 22, 2008, all of which are hereby incorporated by reference herein.

In some embodiments various components of the IOL, such as the anterior element, the intermediate layer, and the posterior element, can be made from one or more suitable polymeric compositions. In some embodiments the optic components are made of substantially the same polymeric material. Exemplary polymeric compositions that can be used for components of the IOL include those described in commonly owned, co-pending U.S. patent application Ser. No. 12/034,942, filed Feb. 21, 2008, and U.S. patent application Ser. No. 12/177,720, filed Jul. 22, 2008. “Flowable media” as used herein includes, but is not limited to, silicone oils. All of the components of the optic portion, including the active flowable media and the passive flowable media, can be substantially index-matched to provide for a generally singular lens element defined by the anterior surface of the anterior element and the posterior surface of the posterior element. “Substantially index-matched” as used herein refers to an IOL whose components are intended to have the same index of refraction and those whose components have indices of refraction that are substantially equal. Some of the components may, however, have different indices of refraction, creating additional interfaces within the IOL.

When it is desirable to index match the materials as closely as possible, two or more silicone oils can be blended together to create a flowable media that has a blended index of refraction that is closer to the index of refraction of a polymer than either of the two or more silicone oils individually. This index-matching technique can be useful when a commercial silicone oil has an index of refraction that is close to, but not as close as desired, the index of refraction of a polymeric composition that is used for components of the IOL. In some embodiments the polymer is chosen with a given refractive index. Two or more fluids are then blended together, at the desired percentages, so that the fluid has an index of refraction that is matched as closely as possible to the index of refraction of the polymer.

An additional technique for enhancing the index matching between fluids (e.g., silicone oil) and polymers in an IOL is to select the polymers and fluid such that the polymers will absorb the fluid (to a certain degree). By absorbing a certain amount of the fluid, the refractive index mismatch between the fluid and the polymer is decreased because the resulting index of refraction of the polymer is closer to the index of refraction of the fluid. After the polymer absorbs some silicone oil, the polymer essentially becomes a polymer-fluid mixture with an index of refraction that is between the refractive index of the polymer and the refractive index of the fluid.

While the disclosure has highlighted designing or selecting a lens to account for different capsule sizes and/or changes that occur after implantation, additional post-implant modifications can be used with the lenses described herein. For example, any methods of post-implant modification, or any lens features, described in the following patent applications can be used to adjust the lens after implantation: U.S. application Ser. No. 10/358,038, filed Feb. 2, 2003, U.S. application Ser. No. 10/890,576, filed Oct. 7, 2004, U.S. application Ser. No. 11/507,946, filed Aug. 21, 2006, U.S. application Ser. No. 12/178,304, filed Jul. 23, 2008, U.S. application Ser. No. 10/360,091, filed Feb. 6, 2003, U.S. application Ser. No. 10/639,894, filed Aug. 12, 2003, U.S. application Ser. No. 11/284,068, filed Nov. 21, 2005, U.S. Provisional Application No. 60/402,746, filed Aug. 12, 2002, U.S. Provisional Application No. 60/405,471, filed Aug. 23, 2002, U.S. Provisional Application No. 60/487,541, and U.S. application Ser. No. 10/231,433, filed Aug. 29, 2002, all of which are incorporated by reference herein. It may be advantageous not only to be able to select an appropriately sized IOL as described herein, but to modify the lens after implant. A post-implant adjustment to any of the lenses described herein can be used to, for example, adjust the set-point of the lens after implantation.

While the disclosure herein highlighted a specific structure characteristic of a fluid-filled AIOL which can be used to account for the variability in capsule size and for changes that occur within the eye or to the lens, the disclosure is not intended to be so limited. Alternative AIOLs (including fluid-driven and non-fluid driven) can similarly be configured and arranged to have a non-linear responses to capsular bag forces, varying power rate changes, etc. As described herein, it may be highly beneficial for any or all AIOLs, after implantation, to be able to deform in response to capsular contraction without (or with very little) change in the optical power of the lens.

Exemplary alternative AIOLs that can be modified to account for changes that occur within the eye after implantation are described in U.S. Pat. No. 7,452,378, U.S. Pat. No. 7,452,362, U.S. Pat. No. 7,238,201, U.S. Pat. No. 7,226,478, U.S. Pat. No. 7,198,640, U.S. Pat. No. 7,118,596, U.S. Pat. No. 7,087,080, U.S. Pat. No. 7,041,134, U.S. Pat. No. 6,899,732, U.S. Pat. No. 6,884,261, U.S. Pat. No. 6,858,040, U.S. Pat. No. 6,846,326, U.S. Pat. No. 6,818,158, U.S. Pat. No. 6,786,934, U.S. Pat. No. 6,764,511, U.S. Pat. No. 6,761,737, U.S. Patent Application Publication No. 2008/0269887, U.S. Pat. No. 7,220,279, U.S. Patent Application Publication No. 2008/0300680, U.S. Patent Application Publication No. 2008/0004699, U.S. Patent Application Publication No. 2007/0244561, and U.S. Patent Application Publication No. 2006/0069433, all of which are hereby incorporated by reference herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An accommodating intraocular lens (“AIOL”), comprising:
an optic portion and a peripheral portion; and
a fluid disposed within at least one of the optic portion and the peripheral portion,
wherein the AIOL has a non-linear power response to increasing amounts of capsular force.

2. The AIOL of claim 1 wherein a power change of the AIOL during a second portion of the non-linear response is substantially greater than a power change of the AIOL during a first portion of the non-linear response.

3. The AIOL of claim 2 wherein the power change during the first portion of the non-linear response is greater than zero.

4. The AIOL of claim 1 wherein the power change of the AIOL during a first portion of the non-linear response is substantially zero.

5. The AIOL of claim 1 wherein the peripheral portion defines a fluid chamber in fluid communication with a fluid channel within the optic portion, wherein the fluid is disposed within the fluid chamber and the fluid channel.

6. The AIOL of claim 5 wherein the fluid pressure in the active channel increases from a first pressure to a second pressure during a first portion of the non-linear response, and wherein the fluid pressure increases from the second pressure to a third pressure during a second portion of the non-linear response.

7. The AIOL of claim 5 wherein the fluid pressure in the active channel remains substantially the same during the first portion and wherein the fluid pressure increases from a first pressure to a second pressure during the second portion.

8. The AIOL of claim 1 wherein the optic portion comprises an anterior element, a posterior element, and an intermediate element disposed between the anterior element and the posterior element, wherein the intermediate element deflects in response to capsular forces.

9. The AIOL of claim 8 wherein the intermediate element comprises an actuator which deflects in response to capsular forces on the AIOL.

10. The AIOL of claim 8 wherein the intermediate element deflects during a first portion of the non-linear response and during a second portion of the non-linear response.

11. The AIOL of claim 10 wherein the intermediate element is not in contact with the anterior element at the beginning of the first portion, and is in contact with the anterior element at the beginning of the second portion.

12. The AIOL of claim 8 wherein a curvature of the anterior element changes more during the second portion than during the first portion.

13. The AIOL of claim 8 wherein intermediate element and posterior element define an active channel in fluid communication with the peripheral portion, and wherein the anterior element and the intermediate element define a passive chamber, wherein the fluid is a first fluid disposed within the active channel and peripheral portion, and wherein the passive chamber contains a second fluid.

14. The AIOL of claim 1 wherein the peripheral portion comprises a haptic which deforms in response to capsular bag forces.

15. An accommodating intraocular lens (“AIOL”), comprising:
an optic portion and a non-optic portion peripheral to the optic portion,
wherein the optic portion comprises an actuation element that changes configuration in response to capsular forces on the AIOL, and
wherein the AIOL has a non-linear power response in response to capsular forces on the AIOL.

16. The AIOL of claim 15 wherein the optic portion comprises an anterior element and a posterior element, wherein the actuation element is disposed between the anterior element and the posterior element.

17. The AIOL of claim 16 wherein the actuation element is not in contact with the anterior element at the beginning of a first portion of the non-linear response.

18. The AIOL of claim 17 wherein the actuation element is in contact with the anterior element at the beginning of a second portion of the non-linear response.

19. The AIOL of claim 16 wherein the curvature of the anterior element is adapted to deform in response to capsular forces, and wherein the curvature of the anterior element deforms more during a second portion of the non-linear response than during a first portion of the non-linear response.

20. The AIOL of claim 15 wherein the power of the AIOL changes substantially less during a first portion of the non-linear response than during a second portion of the non-linear response.

21. The AIOL of claim 15 where the power of the AIOL during a first portion of the non-linear response remains substantially constant.

22. The AIOL of claim 15 wherein the AIOL further comprises a fluid disposed within at least one of the optic portion and the peripheral portion, wherein the actuation element is adapted to change configuration in response to fluid displacement within the AIOL.

23. A method of accounting for capsular forces on an accommodating intraocular lens, comprising:
providing an accommodating intraocular lens (“AIOL”) with an optic portion and a peripheral portion;
implanting the AIOL within an eye; and
allowing the AIOL to have a non-linear power response to capsular forces on the AIOL while allowing an actuation element within the optic portion to change configurations.

24. The method of claim 23 wherein allowing an actuation element within the optic portion to change configurations comprises allowing an actuation element disposed between an anterior element of the optic portion and a posterior element of the optic portion to deflect towards the anterior element or the posterior element.

25. The method of claim 24 wherein the allowing step comprises allowing the actuation element to move towards the anterior element or the posterior element without engaging the anterior element or the posterior element during a first portion of the non-linear response.

26. The method of claim 25 wherein the allowing step comprises allowing the actuation element to engage the anterior element or the posterior element during a second portion of the non-linear response.

27. The method of claim 23 wherein providing a plurality of power change phases in response to capsular forces on the AIOL comprises providing a first portion of the non-linear response during which the power of the AIOL changes substantially less than during a second portion of the non-linear response.

28. The method of claim 27 wherein the power of the AIOL remains substantially the same during the first portion of the non-linear response.

29. The method of claim 23 wherein the allowing step comprises allowing a curvature of an anterior element to change more during a second portion of the non-linear response than during a first portion of the non-linear response.

30. A kit of accommodating intraocular lenses, comprising:
a plurality of accommodating intraocular lenses each of which comprises an optic portion and a peripheral portion, wherein each of the plurality of accommodating intraocular lenses comprises an optic portion element with a different physical parameter.

31. The kit of claim 30 wherein the different physical parameter comprises a dimension of the optic portion component.

32. The kit of claim 31 wherein the optic portion component is an actuator disposed between an anterior surface and a posterior surface of the optic portion.

33. Method of selecting an accommodating intraocular lens for implantation;
measuring a capsular bag characteristic;
selecting an accommodating intraocular lens, based at least in part on the measured characteristic, from a plurality of accommodating intraocular lenses, wherein each of the accommodating intraocular lenses has an optical portion element with a different physical parameter;
implanting the accommodating intraocular lens within a patient's eye.

34. The method of claim 33 wherein the selecting step comprises selecting an accommodating intraocular lens with the physical parameter which will provide a non-linear power response to capsular forces on the intraocular lens.

35. A method of accommodating an intraocular lens (“AIOL”), comprising:
providing an AIOL that changes power during a first portion of a non-linear power response to a first type of ciliary muscle movement, and wherein the AIOL changes power during a second portion of the non-linear response to a second type of ciliary muscle movement, wherein the first and second types of ciliary muscle movements are the same types of movement, and wherein the power change during the first portion of the non-linear power response is different than the power change during the second portion of the non-linear power response; and
implanting the accommodating intraocular lens within a patient's eye to provide an implanted AIOL with a non-linear power response.

36. The method of claim 35 wherein the power change during the first portion is substantially less than during the second portion, and wherein the first portion occurs before the second portion.

37. The method of claim 36 wherein there is substantially no power change during the first portion.

38. The method of claim 35 wherein the AIOL comprises a surface element, and wherein the degree of change in the curvature of the surface element during the first portion is different that the degree of change in curvature of the surface element during the second portion.

39. The method of claim 35 wherein the first and second types of ciliary muscle movement are ciliary muscle contraction.

40. A method of accommodating an accommodating intraocular lens (“AIOL”), comprising:
providing an AIOL that has a non-linear power change response to a singular type of ciliary muscle movement;
implanting the AIOL in a patient's eye;
allowing the AIOL to accommodate in response to the singular type of ciliary muscle movement in a non-linear manner.

41. The method of claim 40 wherein the singular type of ciliary muscle movement is ciliary muscle contraction.

42. An accommodating intraocular lens, comprising:
an optic portion and a peripheral portion, wherein the accommodating intraocular lens has a non-linear power change response to a singular type of ciliary muscle movement.

43. An accommodating intraocular lens (“AIOL”), comprising:
an optic portion, a peripheral portion, and a fluid disposed within the optic portion and the peripheral portion,
wherein the optic portion and the peripheral portion are in fluid communication,
wherein the peripheral portion deforms in response to capsular reshaping due to ciliary muscle movement to displace the fluid between the peripheral portion and the optic portion, and
wherein the peripheral portion is configured such that substantially no fluid is displaced between the peripheral portion and the optic portion in response to a non-ciliary muscle movement related capsular reshaping.

44. The AIOL of claim 43 wherein the peripheral portion comprises at least one haptic in fluid communication with the optic portion, wherein the haptic is configured to deform in response to non-ciliary muscle movement related capsular reshaping such that substantially no fluid is displaced between the peripheral portion and the optic portion.

45. The AIOL of claim 43 wherein a dimension of the at least one haptic is greater than a dimension of a capsule into which the AIOL is implanted.

46. The AIOL of claim 43 wherein the peripheral portion includes at least one haptic which has an oval cross-sectional shape.

47. A method of delivering a two part accommodating intraocular lens (“AIOL”), comprising
delivering a frame element within a patient's capsule such that the frame engages and reshapes the capsule; and
delivering an AIOL within the capsule to a position to allow the AIOL to accommodate in response to ciliary muscle movement.

48. The method of claim 47 wherein delivering the frame comprises allowing the frame to reconfigure from a delivery configuration to an implanted configuration.

49. The method of claim 47 wherein reconfiguring the capsule comprises extending the capsule in an axially directed direction.

50. The method of claim 49 wherein extending the capsule in an axially directed direction comprises extending an anterior portion of the capsule in an anterior direction and extending a posterior portion of the capsule in a posterior direction.

51. The method of claim 47 wherein delivering the frame element comprises preventing the capsule from applying forces on the AIOL due to non-ciliary muscle movement related capsular forces.

52. The method of claim 47 wherein the method does not include securing the frame element to the AIOL.
